(19) United States
(12) Patent Application Publication
(10) Pub. No.: US 2004/0122354 A1
(43) Pub. Date: Jun. 24, 2004

(54) Infusion catheter having an integrated doppler transducer

(76) Inventor: Charles P. Semba, Palo Alto, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/655,277

(22) Filed: Sep. 3, 2003

Related U.S. Application Data
(60) Provisional application No. 60/408,522, filed on Sep. 5, 2002.

(51) Int. Cl. A61M 31/00

(52) U.S. Cl. 604/66; 600/504

(57) ABSTRACT

Preferred embodiments disclosing an infusion catheter having an integrated Doppler transducer are provided which advance the field by providing improved structures for the real time monitoring of blood flow for use in such fields as thrombolytic therapy. Method of practicing therapy using preferred embodiments of the present invention are also provided.

### CROSS-REFERENCE TO RELATED APPLICATIONS

This is non-provisional application filed under 37 CFR 1.53(b), claiming priority under USC Section 119(e) to provisional Application Serial No. 60/408,522 filed Sep. 5, 2002.

### BACKGROUND OF THE INVENTION

1. Field of the Invention

The invention relates generally to catheter devices for use in thrombolytic therapy and, more specifically, to catheter devices for treating arterial and venous clots. The invention further concerns a method for infusing a lytic agent into blood transferring vessel of a patient and real time monitoring of the degree of blood flow using such catheter devices.

2. Description of the Related Art

Thrombolytic therapy has been a major advance in the treatment of acute myocardial infarction (AMI) and other thrombolytic disorders. In AMI, thrombolytics can re-establish perfusion in occluded arteries, resulting in smaller infarct size, improved left ventricular function, and improved short and long-term survival. See, e.g., Braunwald E., Circulation 79:441-1 (1989); Braunwald, N. Engl. J. Med. 329:1650-2 (1993); The GUSTO investigators. An international randomized trial comparing four thrombolytic strategies for acute myocardial infarction. N. Engl. J. Med. 329:673-82 (1993).

Efficacy of thrombolytic therapy can be improved by catheterization of the thrombosed blood vessel in order to deliver the thrombolytic agent locally to the site of thrombus or directly into the thrombus. Thus, the treatment of arterial and venous clots, such as in the case of deep vein thrombosis and pulmonary embolism, often requires the technique of locally delivering a plasminogen activator through a specialized infusion catheter which spans the clotted blood vessel. This infusion catheter, which possesses multiple holes to allow blood to be dispersed relatively equally throughout the clot catheter, is normally placed under X-ray guidance. Following placement of the catheter (usually in a catheterization lab), the patient is transferred to a hospital ward or intensive care unit for monitoring while undergoing low-dose infusion of a lytic agent. During this infusion, the most serious adverse event related to thrombolytic therapy is a major hemorrhage. The risk of hemorrhage is related to the total dose of plasminogen activator delivered and the total duration of lytic therapy. Consequently, a device that could alert the physician or nurse as to the patency of the treated vessel could allow shorter infusion times and fewer bleeding complications and, furthermore, enhance the overall safety of the procedure. Unfortunately, in current practice, there is no rapid, non-invasive method to assess the patency of the clotted blood vessel without the use of X-ray during infusion therapy.

### SUMMARY OF THE INVENTION

Previously, intravascular devices equipped with ultrasound energy have been used to vibrate and ablate clots. In addition, catheters using ultrasound energy for medical imaging purposes are practiced. However, current practice does not adequately provide for real-time monitoring of blood flow as part of an infusion catheter. Instead, a patient must be subjected to repeated X-rays or further invasive practices, such as the insertion of a monitoring probe.

Preferred embodiments are provided which advance the field by providing improved structures and methods for practicing thrombolytic therapy. In addition, alternate preferred embodiments offer improved structures and methods for practicing other forms of therapy in which it is advantageous to locally infuse a therapeutic agent and then monitor in real time the blood flow within a vessel. The provided embodiments seek to advance the art by providing one or more features including, among others, addressing the aforementioned problems.

In accordance with a preferred embodiment a hollow catheter body having infusion ports therein is provided, including a transducer wire of sufficient length to allow placement of a transducer wire proximate to a desired location within a patient. The transducer wire is partially located inside the hollow catheter body, the transducer wire removably adjoining the hollow catheter body so as to allow the selective insertion of the transducer wire through the hollow catheter body. In addition, an ultrasound transducer is joined to the transducer wire distal tip portion, with the transducer being configured to protrude into blood surrounding the infusion catheter body. The position of the ultrasound transducer allows the detection of the presence of a Doppler signal based on an ultrasound signal generated by the transducer. The transducer is also configured to produce an output signal based on the degree of movement of the surrounding blood. An output device is also provided to allow a health care practitioner to interpret the transducer based signal so as to gain information about the degree of movement of the blood surrounding the ultrasound transducer. In addition, a signal transfer wire, a portion of the wire being located inside the transducer wire, is provided to transfer the output signal from the ultrasound transducer to the output device, such as an acoustic output device or visual output device. A method of using the provided infusion system to monitor the degree of blood flow in a patient subsequent to infusion of a therapeutic agent into a blocked blood vessel is also disclosed.

For purposes of summarizing the invention and the advantages achieved over the prior art, certain objects and advantages of the invention have been described herein above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these embodiments are intended to be within the scope of the invention herein disclosed. These and other embodiments of the present invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

FIG. 1 is a cross-sectional sketch of an infusion catheter having an integrated Doppler probe, in accordance with an embodiment of the present invention;

FIG. 2 is a cross-sectional sketch of an infusion catheter of FIG. 1, the catheter being shown removably connected to a port assembly;

FIG. 3 is a schematic overview sketch showing the relation of the infusion catheter and port assembly of FIG. 2 to a therapeutic agent source and an output device;

FIG. 4A is a cross-sectional sketch of a blood vessel, the catheter of FIG. 3 being shown in a position to practice thrombolytic therapy through treating an adjacent thrombosis;

FIG. 4B is a cross-sectional sketch of the blood vessel and catheter of FIG. 4, the thrombosis having been successfully treated; and

FIG. 5 is a flowchart of a method of practicing thrombolytic therapy, in accordance with an embodiment of the present invention.

### DETAILED DESCRIPTION OF THE PREFERRED EMBODIMENT

Definitions

The terms “thrombolytic agent” and “lytic agent” are used interchangeably, and refer to molecules, usually serine proteases or serine protease variants, that work by converting plasminogen to plasmin. Plasmin lyses blood clots by breaking down the fibrinogen and fibrin found in the clots.

The terms “wild-type human tissue plasminogen activator,” “wild-type human t-PA,” and “wild-type ht-PA” as used herein, refer to human extrinsic (tissue-type) plasminogen activator having fibrinolytic activity that typically has a structure with five domains (finger, growth factor, Kringle-1, Kringle-2, and protease domains). The nucleotide and amino acid sequences of wild-type (native) human t-PA have been reported by Pennica et al., Nature 301:214 (1983) and in U.S. Pat. No. 4,766,075, issued Aug. 23, 1988. The location of a particular amino acid in the polypeptide chain of t-PA is identified by a number. The number refers to the amino acid position in the amino acid sequence of the mature, wild-type human t-PA polypeptide as disclosed in U.S. Pat. No. 4,766,075. In the present application, similarly positioned residues in t-PA variants are designated by these numbers even though the actual residue number is not so numbered due to deletions or insertions in the molecule. This will occur, for example, with deletional or insertional variants. The amino acids are identified using the one-letter or three-letter code. Substituted amino acids are sometimes designated herein by identifying the wild-type amino acid on the left side of the number denoting the position in the polypeptide chain of that amino acid, and identifying the substituted amino acid on the right side of the number. For example, replacement of the amino acid threonine (T) by asparagine (N) at amino acid position 103 of the wild-type human t-PA molecule yields a t-PA variant designated T103N t-PA. Similarly, the t-PA variant obtained by additional substitution of glutamine (Q) for asparagine (N) at amino acid position 117 of the wild-type human t-PA molecule is designated T103N-, N117Q t-PA. Deletional variants are identified by indicating the amino acid residue and position at either end of the deletion, inclusive, and placing the Greek letter delta, “Δ”, to the left of the indicated amino acids. Insertional t-PA variants are designated by the use of brackets “[ ]” around the inserted amino acids, and the location of the insertion is denoted by indicating the position of the amino acid on either side of the insertion.

The various domains within the wild-type human t-PA (ht-PA) amino acid sequence have been designated, starting at the N-terminus of the amino acid sequence of human tissue plasminogen activator, as 1) the finger region (F) that has variously been defined as including amino acid 1 upwards of about 44, 2) the growth factor region (G) that has been variously defined as stretching from about amino acid 45 upwards of amino acid 91 (based upon its homology with EGF), 3) Kringle-1 (K1) that has been defined as stretching from about amino acid 92 to about 173, 4) Kringle-2 (K2) that has been defined as stretching from about amino acid 180 to about amino acid 261 and 5) the so-called (serine) protease domain (P) that generally has been defined as stretching from about amino acid 264 to the C-terminal end of the molecule. These domains are situated contiguously generally of one another, or are separated by short “linker” regions, and account for the entire amino acid sequence from about 1 to 527 amino acids in its putative mature form.

The term “human tissue plasminogen activator variant” or “ht-PA variant” is used to refer to a tissue plasminogen activator, which differs from wild-type ht-PA at at least one amino acid position, and retains a functional fibrin binding region and serine protease domain. The finger (F), growth factor (GF), and (to a lesser extent) Kringle-2 (K2) domains of wild-type ht-PA are known to be involved in fibrin binding. An ht-PA variant having a functional fibrin binding region will retain at least the minimal sequences of these domains that are required for fibrin binding. The serum protease domain is responsible for the enzymatic activity for wild-type ht-PA. An ht-PA variant having a functional serine protease domain retains at least the minimal sequences from the serine protease domain of wild-type ht-PA required for converting plasminogen to plasmin in the presence of a plasma clot or in the presence of fibrin.

The terms “TNK t-PA,” “T103N, N117Q, KHRR(296-299)AAAA t-PA,” “tenecteplase,” and “TNKase™,” are used interchangeably and designate a human t-PA variant, which has a threonine (T) replaced by an asparagine at amino acid position 103, adding a glycosylation site at that position, an asparagine (N) replaced by glutamine at position 117, removing a glycosylation site at that position, and four amino acids, lysine (K), histidine (H), arginine (R), and arginine (R) replaced by four alanines (A,A,A,A) at amino acid positions 296-299 of the wild-type human t-PA amino acid sequence. TNKase™ (Genentech, Inc., South San Francisco, Calif.) has been approved by the FDA for use in the reduction of mortality associated with AMI as a single intravenous bolus.

The term “urokinase” is used in the broadest sense to include wild-type native mature urokinase, pro- and prepro-urokinase of any species, including wild-type human urokinase, po- and prepro-urokinase disclosed, e.g. in Ratzkin et al., Proc. Natl. Aca. Sci. USA 78:331307 (1981); Nagai et al., Gene 36:183-8 (1985), and fragments and variants (including amino acid sequence variants and glycosylation variants) thereof.

The term “streptokinase” is use in the broadest sense to include wild-type native mature streptokinase of any species, including wild-type human streptokinase, and fragments and variants (including amino acid sequence variants and glycosylation variants) thereof. See, e.g. Malke and Ferretti, Proc. Natl. Acad. Sci., USA 81:3557-61 (1984)).

The terms “low molecular weight heparin” and “LMW heparin” are used interchangeably, and refer to heparin fractions typically prepared by fractionation and/or depolymerization of heparin so as to achieve significant reduction in average molecular weight as compared with whole heparin preparations. Compositions containing, procedures for making, and methods for using low molecular weight heparin are described in various patent publications, including U.S. Pat. Nos. 4,281,108, 4,687,765, 5,106,734, 4,977,250, 5,576,304, and EP 372 969, the contents of which are hereby expressly incorporated by reference. LMW heparins for use in the present invention preferably have an average molecular weight of about 10 kD or less, more preferably of about 8 kD or less, most preferably less than about 5 kD. It is further preferred that LMW heparins should be of relatively uniform molecular weight e.g. with at least about 60%, more preferably at least about 80% of polymer units having a molecular weight within the above defined average molecular weight limits.

The expressions “fibrinolytic activity”, “thrombolytic activity” and “clot lysis activity” and “lytic activity” are used interchangeably and refer to the ability of a lytic or thrombolytic agent to lyse a clot, whether derived from purified fibrin or from plasma, using any in vitro clot lysis assay known in the art, such as the purified clot lysis assay by Carlson, R. H. et al., Anal. Biochem. 168, 428-435 (1988) and its modified form described by Bennett, W. F. et al., J. Biol. Chem. 266 5191-5201 (1991).

The term “thrombolytic disorder” is used in the broadest sense and refers to any condition characterized by the formation of a thrombus that obstructs vascular blood flow locally or detaches and embolizes to occlude blood flow downstream (thromboembolism). Thrombolytic disorders specifically include, without limitation, myocardial infarction (MI), venous thrombosis, pulmonary embolism, cerebrovascular accident, arterial embolism, etc.

The term “myocardial infarction” or “MI” is used to refer to ischemic myocardial necrosis usually resulting from abrupt reduction in coronary blood flow to a segment of myocardium. MI is typically a disease of the left ventricle (LV), but damage may extend to the right ventricle (RV) or atria.

The term “venous thrombosis” is used to include all forms of thrombosis, such as thrombosis affecting the superficial veins (superficial thrombophlebitis) and deep vein thrombosis (DVT). Since thrombosis is virtually always accompanied by phlebitis, the terms “thrombosis” and “thrombophlebitis” are used interchangeably.

“Pulmonary embolism” is the sudden lodgment of a blood clot in a pulmonary artery with subsequent obstructed blood supply to the lung parenchyma. The most common type of pulmonary embolus is a thrombus that usually has migrated from a leg or pelvic vein. Most of those that cause serious hemodynamic disturbances form in an iliofemoral vein, either de novo or by propagation from calf vein thrombi. Thromboemboli originate infrequently in the arm veins or in the right cardiac chambers.

The term “cerebrovascular accident” is used to refer to stroke and, in general, infarction due to embolism or thrombosis of intracranial or extracranial arteries, and associated hemorrhage.

The term “antithrombotic therapy” refers to therapy aimed at preventing the formation or growth of a blood clot, or partial or complete dissolution of a blood clot already formed.

“Angiogenesis,” i.e. the growth of new capillary blood vessels, is a multi-step process involving capillary endothelial cell proliferation, migration and tissue penetration, and is crucial to normal tissue formation and repair. Factors that promote angiogenesis are called “angiogenic factors.” A number of known growth factors, including basic and acidic fibroblast growth factor (FGF), transforming growth factor-α (TGF-α), and epidermal growth factor (EGF), are broadly mitogenic for a variety of cell types as well as being angiogenic and are, therefore, potentially useful in promoting tissue repair.

“Vascular endothelial growth factor” (VEGF) is a secreted endothelial cell mitogen that, when delivered in vivo, promotes new blood vessel formation. The VEGF protein consists of two polypeptide chains, linked by two disulfide bonds. Although the protein is generally described as a homodimer, heterodimeric species have also been reported. Through alternative splicing of the VEGF RNA transcript, at least five different forms of the monomer chain can be generated, extending 121, 145, 165, 189, and 206 amino acid residues in length. Tischer et al. (1991) J. Biol. Chem. 266:11947-11954; Houck et al. (1991) Mol. Endocrinol. 5:1806-1814; Charnock-Jones et al. (1993) Biol. Reprod. 48:1120-1128; and Neufeld et al. (1996) Cancer Metastasis Rev. 15:153-158. VEGF₁₆₅ and the 121-residue form, VEGF₁₂₁, appear to be the most prevalent forms in vivo.

VEGF is known to stimulate new blood vessel formation by stimulating endothelial cell proliferation and by inducing chemotaxis of endothelial cells. In contrast to other mitogens such as the fibroblast growth factors, VEGF has a much more restricted range of target cell type, and is mitogenic almost exclusively toward endothelial cells. In addition, VEGF has been shown to regulate the expression of other growth factors and biological mediators and may participate in a growth factor cascade that promotes tissue remodeling and repair.

“Gene therapy” refers broadly to treatment methods in which genes are transferred into cells in order to achieve in vivo synthesis of therapeutically effective genetic products, e.g. in order to replace the defective gene in the case of a genetic defect. “Conventional” gene therapy is based on the principle of achieving a lasting cure by a single treatment. However, there is also a need for methods of treatment in which the therapeutically effective DNA (or mRNA) is administered like a drug (“gene therapeutic agent”) once or repeatedly as necessary. Examples of genetically caused diseases in which gene therapy represents a promising approach are hemophilia, beta-thalassaemia and “Severe Combined Immune Deficiency” (SCID), a syndrome caused by the genetically induced absence of the enzyme adenosine deaminase. Other possible applications are in immune regulation, in which humoral or intracellular immunity is achieved by the administration of functional nucleic acid which codes for a secreted protein antigen or for a non-secreted protein antigen, which may be regarded as a vaccination. Other examples of genetic defects in which a nucleic acid which codes for the defective gene can be administered, e.g. in a form individually tailored to the particular requirement, include muscular dystrophy (dystrophin gene), cystic fibrosis (cystic fibrosis transmembrane conductance regulator gene), hypercholesterolemia (LDL receptor gene). Gene therapy methods are also potentially of use when hormones, growth factors or proteins with a cytotoxic or immune-modulating activity are to be synthesized in the body.

Gene therapy also appears promising for the treatment of cancer by administering so-called “cancer vaccines”. In order to increase the immunogenicity of tumor cells, they are altered to render them either more antigenic or to make them produce certain immune modulating substances, e.g. cytokines, in order to trigger an immune response. This is accomplished by transfecting the cells with DNA coding for a cytokine, e.g. IL-2, IL-4, IFN-γ, TNF-α. To date, most gene transfer into autologous tumor cells has been accomplished via retroviral vectors.

The technologies which are hitherto most advanced for the administration of nucleic acids in gene therapy, make use of retroviral systems for transferring genes into the cells. Thus, recombinant viral vectors have been developed to bring about the transfer of genes by using the efficient entry mechanisms of their parent viruses, this strategy was used in the construction of recombinant retroviral and adenoviral vectors in order to achieve a highly efficient gene transfer in vitro and in vivo.

A plurality of viruses affect their entry into the eucaryotic host by means of mechanisms which correspond in principle to the mechanism of receptor-mediated endocytosis. Virus infection based on this mechanism generally begins with the binding of virus particles to receptors on the cell membrane. After this, the virus is internalized into the cell. This internalizing process follows a common route, corresponding to the entrance of physiological ligands or macromolecules into the cell: first of all, the receptors on the cell surface arrange themselves in groups, and the membrane is inverted inwardly and forms a vesicle surrounded by a clathrin coating. After this vesicle has rid itself of its clathrin coat, acidification takes place inside it by means of a proton pump located in the membrane. This triggers the release of the virus from the endosome. Depending on whether the virus has a lipid coat or not, two types of virus release from the endosome were taken into account: in the case of so-called “naked” viruses (e.g. adenovirus, poliovirus, rhinovirus) it was suggested that the low pH causes changes in configuration in virus proteins. This exposes hydrophobic domains which are not accessible at the physiological pH. These domains thus acquire the ability to interact with the endosome membrane and thereby cause the release of the virus genome from the endosome into the cytoplasm. As for viruses with a lipid coat (e.g. vesicular stomatitis virus, Semliki Forest virus, influenza virus) it is presumed that the low pH modifies the structure or configuration of some virus proteins, thereby promoting the fusion of the virus membrane with the endosome membrane. Viruses which penetrate into the cell by means of this mechanism have certain molecular peculiarities which enable them to break up the endosome membrane in order to gain entry into the cytoplasm.

Other viruses, e.g. the coated viruses Sendai, HIV and some strains of Moloney leukaemia virus, or the uncoated viruses SV40 and polyoma, do not need a low pH for penetration into the cell; they can either bring about fusion with the membrane directly on the surface of the cell or they are capable of triggering mechanisms for breaking up the cell membrane or passing through it. It is assumed that the viruses which are independent of pH are also capable of using the endocytosis route.

A “chemotherapeutic agent” is a chemical compound useful in the treatment of cancer. Examples of chemotherapeutic agents include, without limitation, adriamycin, doxorubicin, epirubicin, 5-fluorouracil, cytosine arabinoside (“Ara-V”), cyclophosphamide, thiotepa, busulfan, cytoxin, taxoids, e.g. paclitaxel and doxetaxel, toxotere, methotrexate, cisplatin, melphalan, vinblastine, bleomycin, etoposide, ifosfamide, aminopterin, dactinomycin, mitomycins, esperamicins, 5-FU, 6-thioguanine, 6-mercaptopurine, actinomycin D, VP-16, chlorambucil, melphalan, etc. Also included within this definition are agents that act to regulate or inhibit hormone action on tumors such as tamoxifen and onapristone.

The terms “treat” or “treatment” refer to both therapeutic treatment and prophylactic or preventative measures, wherein the object is to prevent, slow down (lessen), or reverse an undesired physiological change or disorder, such as the formation of a blood clot and the development of other physiological changes associated with the formation of blood clots, e.g. restenosis; reocclusion; hemorrhage; hemodynamic disturbances; pain, arrhythmias, sinus node disturbances, atrioventricular block, etc. associated with MI. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

A “therapeutic agent” refers to any compound or combination of compounds, as applicable, which advances the treatment of a disease or condition by local delivery to the site of action via an infusion catheter.

As used herein, the phrase “effective amount” or “therapeutically effective amount” is intended to include an amount of a compound or combination of compounds, as applicable, to treat a thrombolytic disorder in a mammal, including humans. The combination of compounds may be, but does not have to be, a synergistic combination. “Synergy” as described, for example, by Chou and Talalay, Adv. Enzyme Regul. 22:27-55 (1984), occurs when the effect (in the present case the thrombolytic effect) of the compounds when administered in combination is greater than the additive effect of the compounds when each is administered alone, as a single agent.

The terms “combination,” “combined” and similar expressions, when used in reference to the administration of two or more compounds, mean that the compounds are administered to a subject concurrently. Concurrent administration includes administration at the same time, in the same formulation or separately, and sequential administration in any order or at different points in time so as to provide the desired therapeutic effect.

### DETAILED DESCRIPTION

In one aspect, the present invention concerns a catheter for administration of lytic agents allowing real-time monitoring of blood flow.

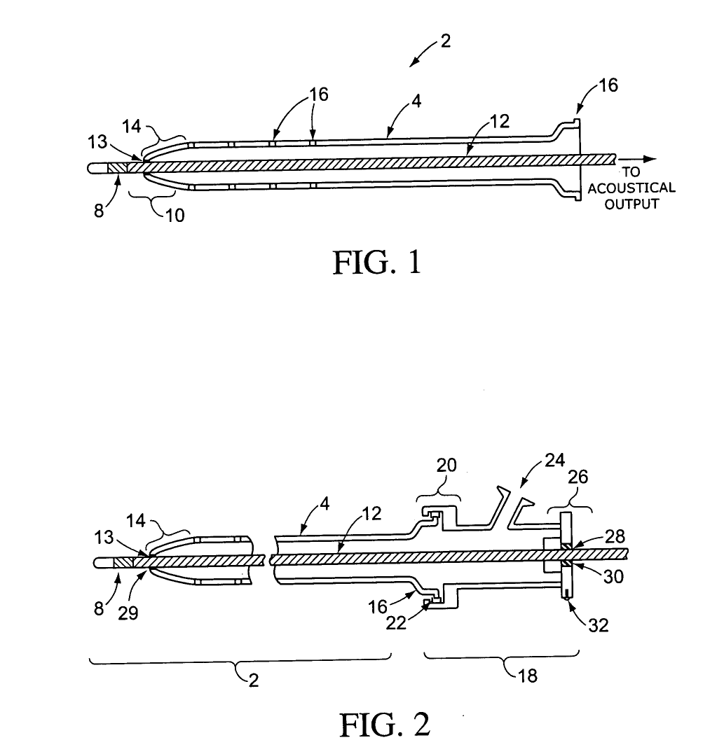

Referring to FIG. 1, a catheter assembly 2 is shown having a hollow infusion catheter body 4, a plurality of infusion ports 6 preferably being located in the walls of the catheter body 4, in accordance with a preferred embodiment. A Doppler transducer 8 is located on the distal tip portion 10 of a transducer wire, the transducer wire 12 being threaded through the interior of the catheter hollow body 4 such that the transducer 8 protrudes from the tapered tip 14 of the catheter body. The transducer wire distal tip portion 10 adjoins the catheter body 4 preferably at the juncture of the transducer wire 12 with the tapered tip 14 so as to allow the selective insertion of the transducer wire through the tapered tip 14 of the hollow catheter body.

A seal 13 is preferably located between the tapered tip 14 and the transducer wire 12. The end of the catheter assembly 2 opposite of the tapered tip 14 is preferably configured to form a hub 16 which allows the catheter assembly 2 to be connected to a port assembly (not shown). The transducer wire 12 is also configured to have conductive wires (not shown) contained within, the wires being operatively connected to the transducer 8 and terminating at an output (not shown).

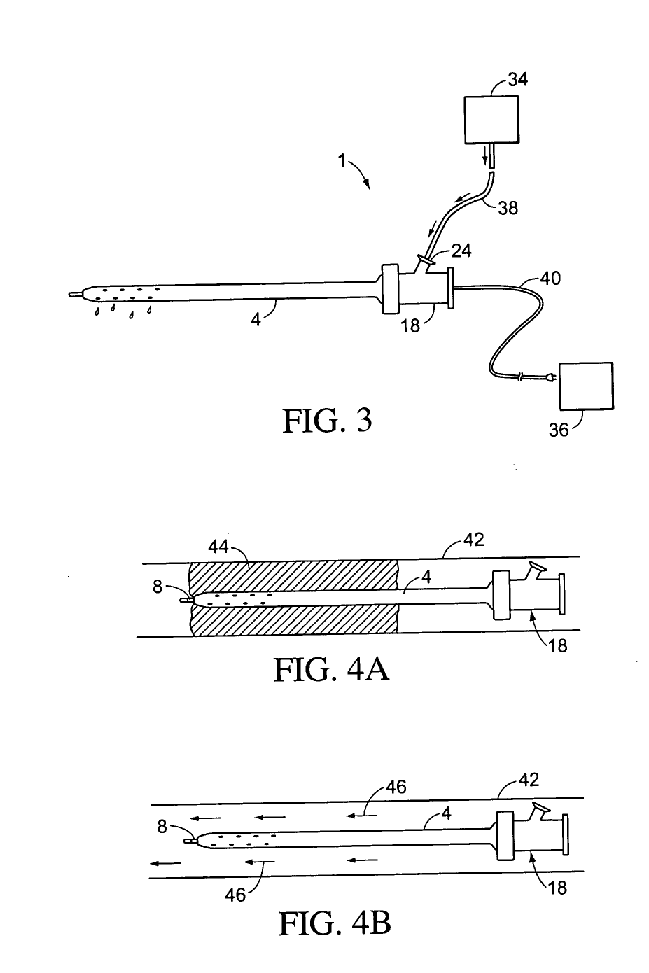

FIG. 2 shows the catheter assembly 2 of FIG. 1 removably connected to a port assembly 18. The port assembly 18 is connected to the catheter assembly hub 16, preferably by an attachment end 20 having a threads 22 which engage the outermost circumference of the hub 16. The connection between the catheter assembly 2 and the port assembly 18 allows the two assemblies to be securely fastened forming a common interior volume, while still allowing the selective separation of the catheter assembly 2 from the port assembly 18 though unscrewing the two assemblies. A therapeutic agent entry port 24 is located in the wall of the port assembly 18 and configured to allow a therapeutic agent, such as a lytic agent, to be introduced into the common interior volume formed by the joined assemblies. The therapeutic agent entry port 24 is preferably configured to allow a tube (FIG. 3) leading to a therapeutic agent source (FIG. 3) to be fastened to the port 24 for the infusion of the therapeutic agent.

In preferred embodiments, the therapeutic agent which is infused through the infusion ports is an thrombolytic or lytic agent which can include any lytic, e.g. thrombolytic agent, known in the art or hereinafter discovered, including, but not limited to, tissue plasminogen activator (t-PA), such as Activase® (Genentech, Inc.), streptokinase, urokinase, t-PA variants, such as rTNK t-PA (tenecteplase, TNKase™, Genentech, Inc.), reteplase (Retavase®, Boehringer Mannheim, GmbH), r-prourokinase, and r-staphylokinase, etc. A sealing mechanism 26 is preferably provided on the end of the port assembly 18 which is not joined with the catheter assembly 2. This sealing mechanism 26, in combination with seal 13, functions to provide a water tight seal around a transducer wire access hole 28 and 29 through which the transducer wire 12 accesses the interior of the port assembly 18 thereby preferably reducing fluid exchange through the access holes 28 and 29. The seal 13 also preferably prevents liquid from leaking in or out of the tapered tip, thereby minimizing non-blood fluid flow proximate to the transducer 8. The water tight seal 30 preferably comprises a silicone diaphragm encircling the transducer wire 12 and a screw 32 which, when tightened, reduces the size of the access hole 28. In alternate preferred embodiments, the junction of the transducer wire 12 and the sealing mechanism 26, in combination with the juncture of the seal 13 at the tapered tip 14 with the transducer wire 12, operatively attaches the transducer wire 12 to the catheter body 4 so that manipulation of the transducer wire 12 allows manipulation of the catheter body 4.

FIG. 3 illustrates the catheter assembly 2 and port assembly 18 shown in FIG. 2 as part of a infusion system 1 including a therapeutic agent source 34 and an output device 36. The therapeutic agent source 34, containing a therapeutic agent, such as a lytic agent, is fluidly connected by a tube 38 to the therapeutic agent entry port 24. The output device 36, such as an acoustic output or amplified speaker, is electrically connected to the Doppler transducer 8 by a signal transfer wire 40, a section of which is routed inside the transducer wire 12 (FIG. 2). In alternate arrangements of the preferred embodiments the output device 36 is a visual output device, which indicates the intensity of the Doppler signal having visual indicators such as one or more indicator lamps which are illuminated to indicate the intensity of the Doppler signal.

In other alternate arrangements the visual output device can also be outputted in the form of a waveform on an oscilloscope. In yet other alternate arrangements, an output device is configured to employ both visual and acoustic signals to relay to a health care practitioner the presence or intensity of the Doppler signal. In alternate arrangements of the preferred embodiments, the infusion system is configured to infuse a therapeutic agent such as an angiogenic factors infused to an occluded blood vessel with the therapeutic intent of growing new blood vessels to the tissue supplied by the obstructed vessel. For example, a protein such as recombinant human vascular endothelial growth factor (rhVEGF) (Genentech, Inc.) could be the delivered to an occluded site. As would be recognized by one skilled in the art, although specific examples of therapeutic agents are provided, it should be understood that the present invention has utility in any type of therapy in which a therapeutic agent is infused locally and the real-time monitoring of the flow within the vessel is desirable. Non-limiting examples of the types of therapy with which the utility of preferred embodiments of the present invention would be advantageous include gene therapy or the administration of chemotherapuetic agents, ordinarily used in cancer treatment.

Referring to FIG. 4A, the catheter of FIGS. 2 and 3 is shown inserted into the interior of a blood vessel 42 in order to practice thrombolytic therapy through treating an adjacent thrombosis 44. In the absence of blood flow, no Doppler signal is produced from the ultrasound energy emitted by the transducer 8 and, as a result, the output device 36 (FIG. 3) preferably does not produce an audible or visual indicator based on the intensity of the Doppler signal. This lack of a Doppler signal intensity indicator signal informs a health care practitioner that blood flow has not resumed.

FIG. 4B illustrates the blood vessel 42 and catheter of FIG. 4A, the thrombosis 44 (FIG. 4A) having been successfully treated, thereby allowing blood flow 46 through the previously clotted vessel 42. The illustrated blood flow 46 combined with the ultrasound energy produced by the transducer 8 yields a form of echo, specifically a Doppler signal, which is indicated to a health care provider when processed to yield a visual or audible indicator. The healthcare provider is therefore informed of the renewed blood flow and can take actions to cease or modify the therapy, if appropriate.

In alternate embodiments, the infusion catheter having the integrated Doppler transducer wire is employed to monitor blood flow at a particular site within a blood vessel where the real time monitoring of blood, in addition to the infusion of the therapeutic agent proximate to the monitoring, is advantageous. Alternate embodiments of the present invention provide utility for measuring in real time blood flow at a particular blood vessel site which is not affected by a thromboysis, but rather is experiencing abnormal blood flow from such non-limiting examples as increased or decreased vessel diameter, an increase or decrease in blood supply upstream of the site, etc. The skilled artisan would readily appreciate other applications in which it would be desirable to monitor in real time blood flow using the a single catheter to both monitor blood flow and infuse a therapeutic agent proximate to the blood flow monitor.

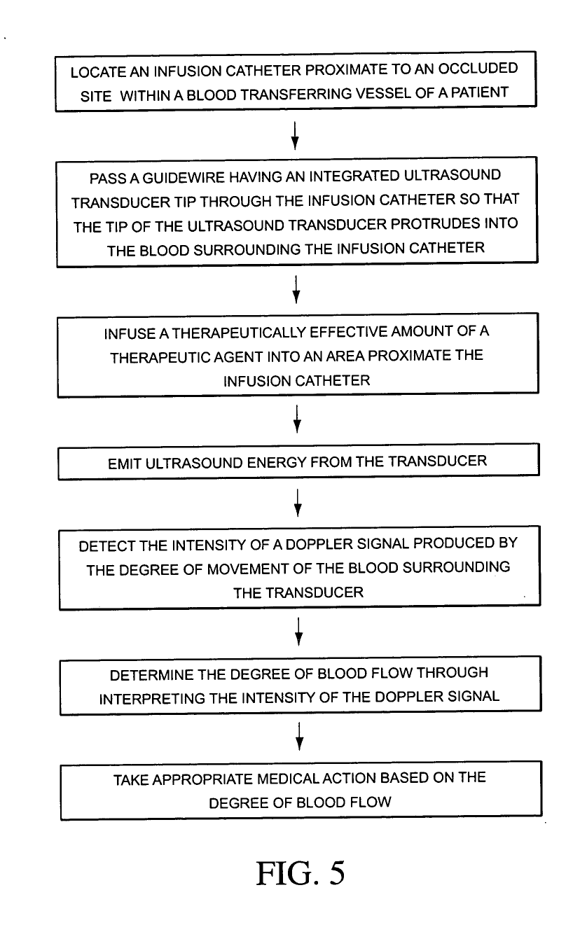

FIG. 5 shows a flowchart of a method of practicing therapy upon an occluded blood vessel using certain preferred embodiments provided herein. An infusion catheter is located proximate to an occluded site within a blood transferring vessel of a patient. Preferably, the infusion catheter is placed in a desired location within the patient by placing a manipulating guidewire in a position which allows a catheter to be passed over the manipulating guidewire to a position adjacent the occluded site. The infusion catheter is then preferably passed over a manipulating guidewire to near the occluded site and preferably the manipulating guidewire is then removed from the infusion catheter. A transducer wire having an integrated ultrasound transducer tip is then passed through the infusion catheter so that the tip of the ultrasound transducer protrudes into the blood surrounding the infusion catheter. A therapeutic agent is then infused into blood surrounding the infusion catheter. Next, ultrasound energy is emitted from the transducer. The degree of blood flow surrounding the transducer is then detected through interpreting the intensity of the Doppler signal, produced by emitting ultrasound energy to indicate the degree of blood movement surrounding the transducer. Appropriate medical action is then preferably taken based on the absence or presence of blood flow. Preferably, the infused therapeutic agent is a lytic agent infused for the purpose of practicing thrombolytic therapy. In alternate embodiments, the infused therapeutic agent is an agent known to produce a beneficial effect on blood flow, e.g. by promoting new blood vessel formation and/or repair of damaged blood vessels or surrounding tissues. In yet other embodiments, the method of FIG. 5 is practiced except the infusion catheter is located proximate to a blood vessel site which is not occluded, but rather experiencing detrimental levels of blood flow and the real time monitoring of blood flow, in addition to the infusion of the therapeutic agent proximate to the monitoring, is advantageous.

The therapeutic agents, such as thrombolytic agents, can be delivered alone or in combination, using the catheter devices of the present invention. In addition, lysis can be facilitated by administration of other pharmaceutical agents, such as heparin, or heparin derivatives, in particular low molecular weight heparin (LMW heparin), in combination with the thrombolytic agent(s).

Low molecular weight heparins (LMWHS) are obtained from standard unfractionated heparin (UFH), and have been used for the prophylaxis and treatment of venous thromboembolism (see, e.g. Schafer, A. I., Hospital Practice Jan. 15, 1997, pp. 99-106). LMWHs have also be used in the treatment of unstable angina and non-Q wave myocardial infarction. Commercially available low molecular weight heparins include, for example LOVENOX® (enoxaparin sodium injection, available from Aventis Pharma Inc. (Bridgewater, N.J.), described in U.S. Pat. No. 5,389,618), FRAGMINM (dalteparin sodium injection, available from Pharmacia, Inc. (Columbus, Ohio)), INNOHEP® (tinzaparin sodium, available from DuPont Pharmaceuticals Company (Wilmington, Del.)), ALPHAPARIN™ (certoparin, available from Alpha, U.K.), FRAXIPARINE™ (nadroparin calcium, available from Sanofi-Synthelabo Canada, Inc.), NORMIFLO™ (ardeparin, available from Wyeth Laboratories, U.S.), and CLIVARINE™ (reviparin sodium, available from ICN Pharmaceuticals).

A particularly advantageous low molecular weight heparin preparation is LOVENOX® (enoxaparin sodium injection), hereinafter referred to as “enoxaparin.” Enoxaparin is a low molecular weight heparin produced by depolymerization of standard unfractionated heparin (UFH). Unlike porcine UFH, which has a molecular weight of 12,000 to 15,000 Daltons, enoxaparin has an average molecular weight of 4,500 Daltons. Compared to UFH, it has more predictable pharmacokinetics, and a higher ratio of anti-Factor Xa to anti-Factor IIa activity. Enoxaparin is also resistant to inactivation by platelet factor 4. In studies examining enoxaparin in acute coronary syndrome patients, enoxaparin has been shown to be safe and more effective than unfractionated heparin at reducing coronary events (Cohen et al., N. Engl. J. Med. 337:447-52 (1997); Antman. E. M. and Women's Hosp., Boston, Mass., Supplement to Circulation 17:504-2649 (1998)).

The administration may take place simultaneously or separately, in any order, using separate formulations or a single formulation, if administration is concurrent. Delivery to the desired location, and penetration into the blood clot can be further enhanced by any method known in the art, such as using vibration, e.g. low frequency vibration.

Formulations of thrombolytic agents, such as t-PA and t-PA variants, are well known in the art and many of them are commercially available. Thus, t-PA variant formulations suitable for catheter delivery include sterile aqueous solutions. Typically, an appropriate amount of a pharmaceutically acceptable salt is also used in the formulation to render the formulation isotonic. A buffer, such as arginine base, in combination with phosphoric acid is also typically included at an appropriate concentration to maintain a suitable pH, generally from about 5.5 to about 7.5. In addition or alternatively, a compound such as glycerol may be included in the formulation to help maintain the shelf-life.

Tenecteplase is currently marketed as a sterile, white to off-white, lyophilized powder for single IV administration after reconstitution with Sterile Water for Injection (SWFI). Each vial of the commercial formulation of tenecteplase (TNKase™) nominally contains 52.5 mg tenecteplase, 0.55 g L-arginine, 0.17 g phosphoric acid, and 4.3 mg polysorbate 20, which includes a 5% overfill, and each vial delivers 50 mg of tenecteplase. The reconstituted solution contains 5 mg/ml tenecteplase. A typical dose of tenecteplase for use with preferred embodiments of the present invention would be 0.25-0.5 mg/hr. However, other pharmaceutical formulations are also specifically within the scope of the present invention.

A typical dose regimen for catheter delivery of wild-type human t-PA (e.g. Alteplase®) is 0.25-1.0 mg/hr. A typical dose of urokinase or streptokinase is 60,000-240,000 U/hr, while a typical dosage of reteplase is 0.25-1.0 U/hr. However, other dosage ranges of these pharmaceutical formulations, in addition to entirely different pharmaceutical formulations, are also specifically within the scope of the present invention. The identification of an effective dose for any particular thrombolytic agent and any particular condition that benefits from the thrombolytic therapy is well within the skill of an ordinary physician.

A feature of preferred embodiments is the enablement of real-time monitoring of the progression of thrombolytic therapy using an infusion catheter without requiring repeated X-rays or additional invasive actions, such as the insertion of an additional monitoring probe. Another feature of preferred embodiment is the reduction of the risk of hemorrhaging through the enabling of a reduction in the duration of thrombolytic therapy by alerting a health care provider as to exactly when thrombolytic therapy is no longer necessary.

Thrombolytic therapy with thrombolytic agents in accordance with the present invention may be combined with the administration of aspirin as early as possible following the thrombotic event, and/or other therapeutic agents, such as β-blockers, calcium channel blockers, angiotensin-converting enzyme (ACE) inhibitors, intravenous nitrates, β-blockers, angiotensin II inhibitors, statins, ticlopidin/clopidogrel, oral anticoagulants, Abciximab, other gpIIb/IIIa inhibitors, angiotensin-receptor blockers, thienopyridines, and thrombolytics, all conventionally used in cardiac treatment.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications thereof. Thus, it is intended that the scope of the present invention herein disclosed should not be limited by the particular disclosed embodiments described above, but should be determined only by a fair reading of the claims that follow.

What is claimed is:

1. An infusion system comprising:
a hollow catheter body having infusion ports therein;
a transducer wire of sufficient length to allow placement of the transducer wire proximate to a desired location within a patient, the transducer wire being partially located inside the hollow catheter body, the remainder of the transducer wire being located outside of the catheter hollow body, a transducer wire distal tip portion removably adjoining the hollow catheter body so as to allow the selective insertion of the transducer wire through the hollow catheter body;
an ultrasound transducer joined to the transducer wire distal tip portion, the transducer being configured, when inserted into blood surrounding the infusion catheter body, to protrude sufficiently to allow the detection of the presence of a Doppler signal based on an ultrasound signal generated by the transducer, the transducer being further configured to produce an output signal based on the degree of blood movement when surrounding by blood; and
an output device being configured to allow a health care practitioner to interpret the transducer based signal in order to gain information about the degree of blood movement proximate to the ultrasound transducer; and
a signal transfer wire, a portion of the wire being located inside the transducer wire, the signal transfer wire being configured to transfer the output signal from the ultrasound transducer to the output device.

2. The infusion system of claim 1, wherein the catheter system is configured for thrombolytic therapy.

3. The infusion system of claim 1, further comprising a port assembly, the port assembly having an attachment end to which the catheter body is removably attached, the transducer wire partially being located within the port assembly, a transducer wire access hole being located opposite the attachment end.

4. The infusion system of claim 3, further comprising a therapeutic agent entry port located on the port assembly.

5. The infusion system of claim 1, wherein the ultrasound transducer protrudes from a distal tip of the hollow catheter body.

6. The infusion system of claim 1, wherein the ultrasound transducer protrudes from a tapered tip of the hollow catheter body.

7. The infusion system of claim 1, wherein the ultrasound transducer is configured to detect a Doppler signal when the blood surrounding the transducer is flowing.

8. The infusion system of claim 7, wherein the output device is an acoustic output device comprising a speaker, a power supply, and appropriate amplification circuitry to allow the output signal generated by the ultrasound transducer to be heard by a human ear when the blood surrounding the transducer is flowing.

9. The infusion system of claim 8, further including a port assembly to which the catheter body is removably attached, wherein the hollow catheter body, the port assembly, and the transducer wire are configured to be inserted into a human blood vessel.

10. The infusion system of claim 7, wherein the output device is a visual output device which produces a visual indicator based on the intensity of the detected Doppler signal.

11. A method of infusion therapy comprising:
locating an infusion catheter and a transducer wire proximate to a treatment site within a blood transferring vessel of a patient, the transducer wire being partially located inside of the infusion catheter, the transducer wire having an integrated ultrasound transducer tip configured to protrude from the infusion catheter into blood surrounding the infusion catheter, when inserted into a blood vessel having blood therein;
infusing a therapeutically effective amount of a therapeutic agent into an area proximate the infusion catheter;
emitting ultrasound energy from the transducer;
detecting the intensity of a Doppler signal produced by the degree of blood flow surrounding the transducer;
determine the degree of blood flow surrounding the transducer through interpreting the intensity of the Doppler signal; and
taking appropriate medical action based on the degree of blood flow.

12. The method of claim 11, wherein locating the infusion catheter and transducer wire proximate to a treatment site within a blood transferring vessel of a patient comprises:
inserting a manipulating guidewire proximate to the treatment site;
passing the infusion catheter over the manipulating guidewire to proximate the treatment site, the catheter having an opening at both ends;
removing the manipulating guidewire from inside of the infusion catheter by removing the guidewire from both of the infusion catheter openings;
guiding a transducer wire, having a Doppler transducer attached to a distal tip portion of the wire, inside the hollow infusion catheter so that the transducer protrudes from the end of the infusion catheter into the surrounding blood.

13. The method of claim 12, further including removing the catheter in response to the Doppler signal.

14. The method of claim 12, wherein the infused therapeutic agent is a lytic agent.

15. The method of claim 14, wherein the lytic agent is a thrombolytic agent selected from the group consisting of tissue plasminogen activator (t-PA), T103N, N117Q, KHRR(296-299)AAAA t-PA, steptokinase, urokinase, reteplase (133-L-serine-174-L-tyrosine-175-L-glutamine-173-527 t_PA), and staphylokinase.

16. The method of claim 15, wherein the thrombolytic agent is native human tissue plasminogen activator (ht-PA) or a variant thereof.

17. The method of claim 16, wherein the tissue plasminogen activator is recombinant native human tissue plasminogen activator (rht-PA).

18. The method of claim 16, wherein the tissue plasminogen activator is T103N, N117Q, KHRR(296-299)AAAA t-PA (tenecteplase).

19. The method of claim 16, wherein the tissue plasminogen activator is co-administered with heparin.

20. The method of claim 19, wherein the heparin is a low molecular weight heparin.

21. The method of claim 12, wherein the infused therapeutic agent is a nucleic acid encoding a polypeptide capable of modifying blood flow proximate an occluded vessel.

22. The method of claim 12, wherein the infused therapeutic agent is an angiogenic factor.

23. The method of claim 22, wherein the angiogenic factor is recombinant human vascular endothelial growth factor (rhVEGF).
